(19) United States
(12) Patent Application Publication
(10) Pub. No.: US 2013/0218039 A1
(43) Pub. Date: Aug. 22, 2013

(54) System and Method for Assessing Breathing

(71) Applicants: John G. Sotos, Palo Alto, CA (US); John L. Branscum, JR., Belmont, CA (US); Patricia H. Branscum, Belmont, CA (US)

(72) Inventors: John G. Sotos, Palo Alto, CA (US); John L. Branscum, JR., Belmont, CA (US); Patricia H. Branscum, Belmont, CA (US)

(73) Assignee: Apneos Corp., Palo Alto, CA (US)

(21) Appl. No.: 13/400,113

(22) Filed: Feb. 19, 2012

(51) Int. Cl. A61B 7/00; A61B 5/08

(52) U.S. Cl. 600/529

(57) ABSTRACT

A system for recording one or more parameters of a patient, comprising a peri-tracheal microphone, a memory, a controller, a power source, and a housing, is disclosed. The microphone provides a signal indicative of at least quiet breathing of the patient. The memory stores information derived from the signal indicative of at least quiet breathing of the patient. The controller writes the information into the memory. The power source supplies electrical energy to at least one of the microphone, the memory, or the controller. The housing contains one or more of the controller or the memory or the power source, and is coupled to a non-peri-tracheal portion of the patient's body such that the housing moves substantially in concert with said non-peri-tracheal portion of the body. Information stored in the memory may be assessed to characterize the state of the patient, possibly including aspects of breathing while awake or asleep.

### CROSS-REFERENCES TO RELATED APPLICATIONS

This application claims priority to U.S. Provisional Patent No. 60/557,735 filed Mar. 30, 2004, commonly assigned, and hereby incorporated by reference for all purposes.

This application claims priority to U.S. Provisional Patent No. 60/580,219 filed Jun. 16, 2004, commonly assigned, and hereby incorporated by reference for all purposes.

This application claims priority to U.S. Provisional Patent No. 60/580,218 filed Jun. 16, 2004, commonly assigned, and hereby incorporated by reference for all purposes.

This application is a continuation-in-part of application Ser. No. 10/721,115 filed Nov. 24, 2003 and commonly assigned.

This application is a continuation-in-part of application Ser. No. 11/094,911 filed Mar. 30, 2005 and commonly assigned.

### BACKGROUND OF THE INVENTION

1. Field of the Invention

The present invention generally relates to ways of characterizing health related disorders. More particularly, a system and method for assessing breathing in a mammal that sleeps is described.

2. Description of the Relevant Art

Sleep-related breathing disorders including, but not limited to, sleep apnea and snoring afflict many people. The diagnosis of such disorders has traditionally been made using a technique called “attended polysomnography” that records a plurality of physiological parameters from a patient while he or she sleeps. Most persons who undergo attended polysomnography (PSG) do so away from home, in a facility called a sleep laboratory and are generally monitored (“attended”) by a trained technician while sleeping there.

It is widely acknowledged that attended polysomnography is both inconvenient for the patient and expensive. As Ross et al (Sleep. 2000; 23:519-532) note: “The development of simpler and less costly alternatives for diagnosis or pre-PSG screening is highly desirable.” Thus, there has been an effort to develop “home sleep diagnostic” techniques for diagnosing sleep-related breathing disorders in the patient's home without a human attendant therein.

In developing a home sleep diagnostic device, there often arises a question of what physiological parameters the diagnostic should measure. Douglas (Sleep Med Rev. 2003;7:53-59) remarks: “The choice of sensors to be used is open to considerable debate.” Several guidelines may be considered when deciding which sensor(s) to include, although none of these are mandatory:

Non-invasive sensors are generally preferable.

Comfortable sensors are generally preferable.

Inexpensive sensors are generally preferable.

Sensors that are easily applied are generally preferable.

Sensors that stay reliably in place are generally preferable.

Sensing highly informative physiological parameters is generally preferable.

Other sensor-related preferences exist. For example, because “some children will not tolerate electrodes applied to the head and face” (Morielli et al. Chest. 1996; 109:680-687), a home sleep diagnostic which has no sensors on the head or face may be preferable, especially when children are expected to be subjects.

Additional considerations may be applied in designing non-sensor portion(s) of a home diagnostic device. For example, many home diagnostic devices include a recording means to store data acquired by the device's sensor(s). The recording means should be reliable and should have sufficient capacity so that data quality or quantity are not compromised. Indeed, a committee writing on behalf of the American Academy of Sleep Medicine has stated “Portable sleep-apnea devices must record raw (unprocessed) data, and stored data must be reproducible” (Thorpy et al. Sleep. 1994; 17:372-377). It is also preferable that a sleep diagnostic device not interfere with sleep.

With so many possible considerations and so many possible sensor types, a plurality of proposed home diagnostics have been proposed. Review articles on home sleep diagnostic devices include: Ferber et al (Sleep. 1994; 17:378-392), Douglas (supra.), Ross et al (supra.), Li and Flemons (Clin Chest Med. 2003; 24:283-295), and Flemons (Chest. 2003; 124:1543-79 and 14 pages of supplementary material published online)

Home sleep diagnostics have included sensor(s) measuring blood oxygen levels (Netzer et al. Chest. 2001; 120:625-633), oronasal airflow (as taught, for example in U.S. Pat. No 6,306,088), body motion, and peripheral arterial tone (as taught, for example, in U.S. Pat. Nos. 6,319,205 and 6,322,515) (Bar et al. Chest. 2003; 123:695-703).

Sound is also a physiological parameter that has been used by home sleep diagnostic techniques. Among the reasons it may be considered an attractive parameter in diagnosis is that sound sensors (microphones) are often widely available inexpensively, and because some indicators of sleep breathing, e.g. snoring, are sonic in nature.

Many persons having a specific type of sleep apnea called obstructive sleep apnea (OSA) emit snoring sounds. Thus, some home sleep diagnostic devices monitor snoring sounds for purposes of diagnosing OSA, e.g. those apparently related to SnoreSat™ diagnostics (Issa et al. Am Rev Respir Dis. 1993; 148:1023-1029) (Issa et al. Sleep. 1993; 16:532) (ComfortAcrylics.com. Internet document, 2003) (Sagatech.ca. Internet document, 2005), those apparently related to MESAM diagnostics (U.S. Pat. Nos. 4,982,738 and 5,275,159) (Stoohs and Guilleminault. Eur Respir J. 1990; 3:823-829) (Penzel et al. Sleep. 1990; 13:175-182), and U.S. Pat. No. 6,811,538 of Westbrook et al.

Snoring sounds are, however, often limited in their diagnostic usefulness because snoring may not be present in all patients with OSA. Furthermore, another type of sleep apnea called central sleep apnea (CSA) is typically less frequently associated with snoring than is OSA.

Thus, other types and sources of sound have been used in the assessment of sleep breathing. U.S. Pat. Nos. 5,797,852 and 6,290,654 teach a microphone positioned near the head.

Some home sleep diagnostics capture sound information from one or more sensors positioned on the upper lip or nearby facial structures, e.g. those associated with SNAP Laboratories LLC (U.S. Pat. Nos. 5,671,733 and 5,782,240 and 5,879,313 and 5,961,447 and 6,045,514) (SNAP Laboratories LLC. Internet document, 2002) and those associated with Sleep Solutions Inc. (U.S. Pat. Nos. 6,171,258 and 6,213,955) (Sleep Solutions Inc. Internet document, 2002). However, because of their facial attachment, these diagnostics may, as noted above, not be accepted by children. Additionally, persons with a mustache may encounter difficulty in achieving or maintaining reliable placement of a sensor about the upper lip.

In virtually all persons, breathing is associated with movement of air in the trachea. This air movement is generally associated with sound production. With suitable equipment and technique, for example a stethoscope applied to a patient's suprasternal notch in a quiet room, tracheal breath sounds are frequently audible during wakefulness and sleep.

Of course, tracheal sound may include sounds transmitted to the trachea, not solely sounds produced there.

Numerous types of physiological events may have tracheal sound manifestations including snoring, coughing, talking, sighing, wheezing, sneezing, yawning, snorting, and the like. Cardiovascular sounds may be present in tracheal sound. Herein we use the term “tracheal breath sound” or “normal tracheal breath sounds” to refer to the component of tracheal sound associated with normal tidal breathing. This definition excludes, for example, snoring, sighing, and yawning, even though they are very common in the population and may, in at least the cases of sighing and yawning and talking, be encountered in normal persons. Some types of apparatus designed to detect relatively loud sounds such as typical snoring and snorting may be unable to detect relatively quiet sounds such as normal tracheal breath sounds.

Tracheal sound may change in association with apnea or other types of hypoventilation. For example, several investigators have found that tracheal breath sounds may vanish or significantly diminish during periods of apnea (Krumpe and Cummiskey. Am Rev Respir Dis. 1980; 122:797-801) (Cummiskey et al. Am Rev Respir Dis. 1982; 126:221-224) (East and East. Comput Meth Prog Biomedicine. 1985; 21:213-220) (Peirick and Shepard. Med Biol Eng Comput. 1983; 21:632-635) (Meslier et al. Sleep. 2002; 25:753-7).

Thus, capture and assessment of tracheal breath sounds have been incorporated into home sleep diagnostics.

Potsic and colleagues (Potsic. Laryngoscope. 1987; 97:1430-1437) (Potsic. Otolaryngol Head Neck Surg. 1986; 94:476-480) (Marsh et al. Otolaryngol Head Neck Surg. 1983; 91:584-585) teach a sound sensor placed in the suprasternal notch (which overlays the trachea in most people). The sensed sound is recorded on an audio cassette tape in a recorder. A potential shortcoming of this device is the storage of tracheal breath sound data in analog format on a component with moving parts, raising concerns about mechanical durability and data fidelity. Another potential shortcoming of this device may be the size of the recording unit and its propensity to impart tension to the sound sensor. Although the size of the recording unit is not disclosed in the cited art, a container measuring 4×6×10 inches was used to mail the device. This suggests the recording unit was of a considerable size and probably large enough that it was not closely coupled to the patient's body, but instead positioned on the same sleeping surface as the patient where its mass (and intertia) may have created a tethering condition with respect to the sound sensor.

U.S. Pat. No. 6,120,441 (to Griebel) teaches a multi-sensor system for recording physiological data from a patient during sleep. Of the eight or nine sensors, one is an electret microphone positioned at the patient's larynx (which is close to the trachea) to pick up “the patient's respiratory sounds and snoring sounds.” Respiratory sounds are digitized, rectified, and filtered before storage in a recording unit. An additional “distribution box” is employed to house certain sensors and receive wires from other sensors. In some cases it appears that patients sleep with the recording unit connected to a computer. A potential shortcomings of this device is its numerous sensors and its complexity. A further potential shortcoming of this device derives the processing performed on the laryngeal sound signal and from a statement by the American Academy of Sleep Medicine, which advises that “portable sleep-apnea [diagnostic] devices must record raw (unprocessed) data” (Thorpy et al. Sleep. 1994; 17:372-377). Another potential shortcoming of this device is the lack of specificity for how the recording unit and, to a lesser degree, the distribution box are mechanically coupled to the patient. The cited art does not disclose a coupling that would minimize the potential for heavier components of the device to create a tethering condition with respect to the sensors.

Hida et al (Tohoku J Exp Med. 1988; 156 (Suppl.):137-142) teach a tracheal microphone, nasal flow sensor, and electrocardiographic sensing means coupled to a recorder weighing about 800 grams. The recorder stores data derived from these sensors, including an envelope signal derived from tracheal sound, onto a cassette tape. A potential shortcoming of this device is that it apparently does not store raw (unprocessed) tracheal breath sound data. An additional potential shortcoming of this device is the storage of tracheal breath sound data in analog format on a component with moving parts, raising concerns about mechanical durability and data fidelity. A further potential shortcoming of this device is related to the weight of the recording unit. Although the size of the recording unit is not provided in the cited art, the weight suggests a size that may have been too large to conveniently couple to the body. The cited art does not provide a description of a mechanical coupling between the recorder unit and the body, thus it seems likely that the recording unit was positioned on the same sleeping surface as the patient, where it may have created a tethering condition with respect to one or more of the sensors.

Hida et al (Respiration. 1993; 60:332-337) teach a tracheal microphone, nasal flow sensor, and electrocardiographic sensing means coupled to a recorder weighing about 280 grams. The recorder stores data derived from these sensors, including an envelope signal derived from tracheal sound, in a storage means. A potential shortcoming of this device is that it apparently does not store raw (unprocessed) tracheal breath sound data. Additionally, the cited art does not describe the size of the recorder nor describe a method for mechanically coupling it to the patient. Thus, a potential shortcoming of this device is that it may have been positioned on the same sleeping surface as the patient where it may have created a tethering condition with respect to the sensors.

U.S. Pat. Nos. 6,168,568 and 6,261,238 (to Gavriely) teach a phonopneumograph (PPG) system that includes a plurality of breath related sensors placed around the respiratory system of a patient for measuring breath related activity. A preferred embodiment of the PPG system includes at least one of the following type of sensors: chest expansion sensors, breath sounds sensors, tracheal sound sensors, flow meters (e.g. pneumotachograph) and spirometers. A preferred embodiment of the PPG system also includes an ambient noise microphone. Various embodiments of the PPG system include additional components, e.g. an electronic stethoscope, a printer, and a monitor. Possible shortcomings of the PPG system as a home diagnostic include its operating complexity, its largely unspecified manner of sensor attachment, the bulk of components such as the printer and monitor, and the effort required to get a PPG system into a patient's home.

U.S. Pat. No. 6,666,830 (to Lehrman and Halleck) teaches one or more collar-mounted microphones positioned adjacent a breathing airway in the patient's neck. The patent further teaches a chest motion sensor coupled to the chest of the patient, an optional airflow sensor near the nostrils, and storage of “signal patterns” derived from the microphone(s). A potential shortcoming of this device is that it apparently does not store raw (unprocessed) tracheal breath sound data. An additional potential shortcoming of this device is the possibility of accidental strangulation from a circumferential band around the neck.

Despite some of the efforts noted above, and others, home sleep diagnostics for disorders such as obstructive sleep apnea have faced difficulty becoming widely adopted, as evidenced by the observation of Li and Flemons (Clin Chest Med. 2003; 24:283-295) that “use of portable monitors at home for managing sleep apnea patients remains controversial and is not currently considered accepted practice by any specialty group.”

From the above, it is desirable to have improved techniques for characterizing health related disorders.

### BRIEF SUMMARY OF THE INVENTION

A system for recording one or more parameters of a patient, possibly including, but not limited to, breathing-related parameters, is disclosed. The system may include a peri-tracheal microphone, a memory, a controller, a power source, and a housing. The microphone provides a signal indicative of at least quiet breathing of the patient. The memory stores information derived from the signal indicative of at least quiet breathing of the patient. The controller writes the information into the memory. The power source supplies electrical energy to at least one of the microphone, the memory, or the controller. The housing contains one or more of the controller or the memory or the power source, and is coupled to a non-peri-tracheal portion of the patient's body such that the housing moves substantially in concert with said non-peri-tracheal portion of the body. Information stored in the memory may be assessed to characterize the state of the patient, possibly including aspects of breathing while awake or asleep.

Various additional objects, features, and advantages of the present invention can be more fully appreciated with reference to the detailed description and accompanying drawings that follow.

### BRIEF DESCRIPTION OF THE DRAWINGS

FIG. 1 shows one embodiment of a system for assessing breathing.

FIGS. 2A and 2B show one embodiment of a wristy.

FIG. 2C shows another embodiment of a wristy.

FIG. 2D shows an embodiment of a wrist unit positioned for use.

FIG. 3 shows an embodiment of a wrist unit.

FIG. 4 shows a block diagram of the circuitry of one embodiment of a wrist unit in a system for assessing breathing.

FIG. 5 shows a flowchart of the operation of one embodiment of a system for assessing breathing.

### DESCRIPTION OF THE SPECIFIC EMBODIMENT

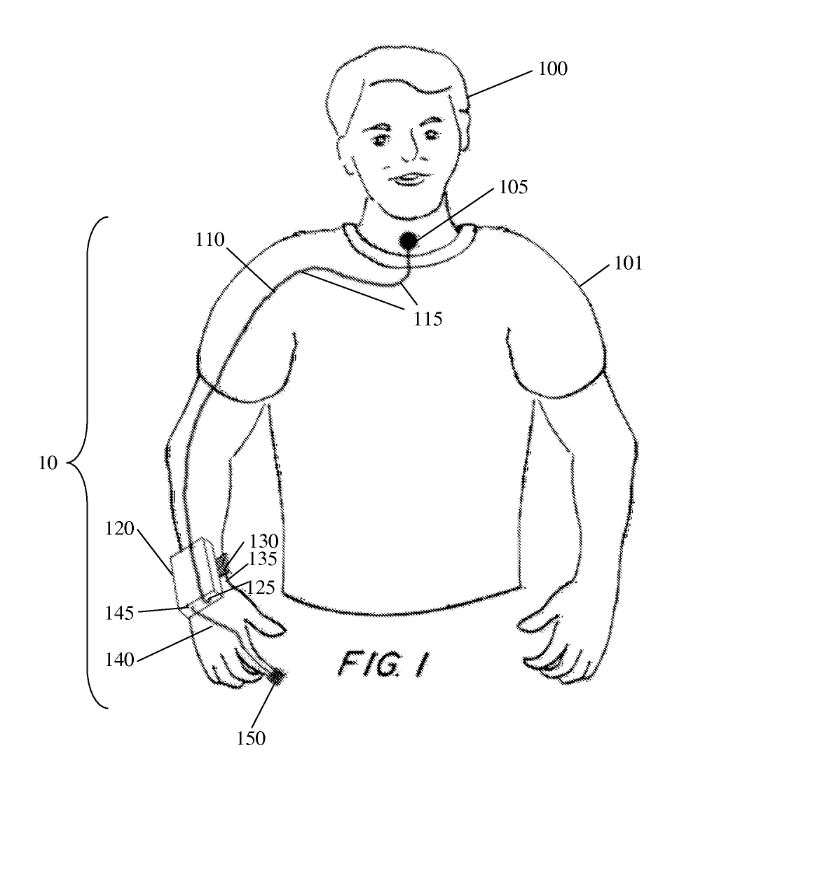

FIG. 1 shows one embodiment of a system 10 for assessing breathing in a patient 100. Patient 100 may wear a nightshirt 101. Nightshirt 101 preferably has a loose collar. In an alternate embodiment, patient 100 wears no shirt.

A tracheal sensor 105 is positioned near a segment of the trachea of patient 100, e.g. in the suprasternal notch or, more generally, between the superior margin of the sternum (manubrium) and the inferior margin of the cricoid cartilage. Alternatively, tracheal sensor 105 may be positioned superficial to the larynx.

In some embodiments, tracheal sensor 105 is removably attached to the skin of patient 100. In one embodiment the attachment is an adhesive means positioned between tracheal sensor 105 and the underlying skin of patient 100. In this case, the adhesive means may be a substantially planar layer, with adhesive on both of its faces. In another embodiment an adhesive means may be positioned superficially to and in contact with tracheal sensor 105 and the nearby skin of patient 100, with adhesive in contact with the nearby skin and possibly tracheal sensor 105. In these embodiments the adhesive means thereby provides a physical coupling of tracheal sensor 105 to patient 100.

In one embodiment, tracheal sensor 105 produces signals indicative of tracheal sounds made by patient 100. This may be done by configuring a microphone (not shown in FIG. 1) within tracheal sensor 105. In one embodiment the microphone is adapted to produce signals indicative of at least quiet (non-snoring) breathing, e.g. tracheal breath sounds.

In one embodiment tracheal sensor 105 includes a microphone and a position-sensing means in a common housing. A more detailed description of this is given in our copending U.S. patent application Ser. No. 10/721,115.

Tracheal sensor 105 is preferably of a size that renders it unobtrusive to patient 100. In one embodiment a cable 110 containing one or more electrically conductive means emanates from tracheal sensor 105 and inserts into a wrist unit 120 at junction point 125. If patient 100 is wearing a night shirt, cable 110 may be affixed to night shirt 101 at one or more fixation points 115. If patient 100 is not wearing a shirt, cable 100 may be affixed to the skin of patient 100 at fixation points 115. In one embodiment, a segment of adhesive tape (not shown) is used to removably affix cable 110 to nightshirt 101 or to the skin of patient 100.

In another embodiment a wireless link comprises at least part of a coupling between one or more components of tracheal sensor 105 and one or more components of wrist unit 120.

In one embodiment, strap 130 removably couples wrist unit 120 to a wrist 135 of patient 100. In another embodiment, strap 130 may pass through a bracket (not shown in FIG. 1) that is substantially rigidly coupled to wrist unit 120. Strap 130 may wrap around wrist 135, fitting snugly but not tightly, thereby providing a comfortable and reasonably rigid mechanical coupling between wrist 135 and wrist unit 120. In one embodiment, strap 130 may have an elastic segment and hook and loop fasteners to facilitate attachment and removal of wrist unit 120.

In other embodiments, wrist unit 120 is coupled to wrist 135 by a glove-like apparatus or by an adhesive. If wrist unit 120 is sufficiently small in size, it may be satisfactorily coupled to a dorsum or other portion of a hand of patient 100.

The mechanical coupling between wrist 135 and wrist unit 130 is often advantageous, as it may help to reduce the frequency with which tracheal sensor 105 may become dislodged or otherwise displaced from its preferred peri-tracheal position. Methods of achieving this coupling include, but are not limited to, using an adhesive means of suitable stickiness; wiping the portion of the skin of patient 100 with an alcohol pad where tracheal sensor 105 is to be adhesively attached, thereby improving adhesion of the sensor 105 to the skin; and minimizing any physical forces applied to tracheal sensor 105 which may tend to dislodge it.

One way to minimize any physical forces impacting tracheal sensor 105 is to reduce its height, making it is less likely to be forced laterally against another object, e.g. a pillow's edge. Alternatively, the forces applied via cable 110 to tracheal sensor 105 may be reduced. Herein the terms “sensor end” and the “free end” refer to the ends of a cable having and not having, respectively, a junction with a sensor.

We have discovered that tethering, wherein a mass exerts tension on a cable by virtue of inertia, is a potential cause of some sensor dislodgements that may occur during sleep. Most people move around while they sleep, and persons sometimes described as “active sleepers” have pronounced sleep-associated movements. Such movements have the potential to result in a tug by a mass at the free end of a cable that is then transmitted to the sensor end, possibly resulting in a force pulling the sensor away from its desired position. For example, if wrist unit 120 were not coupled to wrist 135 but were instead positioned on a night-table next to sleeping patient 100, patient 100 could roll away from the night-table, with the effect that a tension develops in cable 110 (owing to the inertia of wrist unit 120 at the cable's free end) that may pull tracheal sensor 105 off the skin of patient 100. More generally, if a component attached to a cable's free end does not move substantially in concert with the cable's sensor end during certain movements, there is a potential for the component to exert tension on the sensor's adhesion to the patient and dislodge a sensor at the cable's sensor end.

The use of mechanical coupling between wrist 135 and wrist unit 120 in conjunction with a cable 110 of suitable length reduces tethering tendencies because wrist 135 will generally not move beyond a certain fixed distance from tracheal sensor 110, which may be taken as a minimum length of cable 110.

Additionally, a coupling of suitable mechanical rigidity between wrist 135 and wrist unit 120 may permit an acceleration sensor substantially rigidly coupled to wrist unit 120 to provide signals indicative of acceleration of wrist 135.

Cable 110 is preferably of sufficient length to allow patient 100 to freely turn his or her head and move his or her wrist 135 and associated shoulder and arm, without applying undue tension on cable 110. Fixation points 115 are preferably chosen to permit said movement of head and wrist also without undue tension.

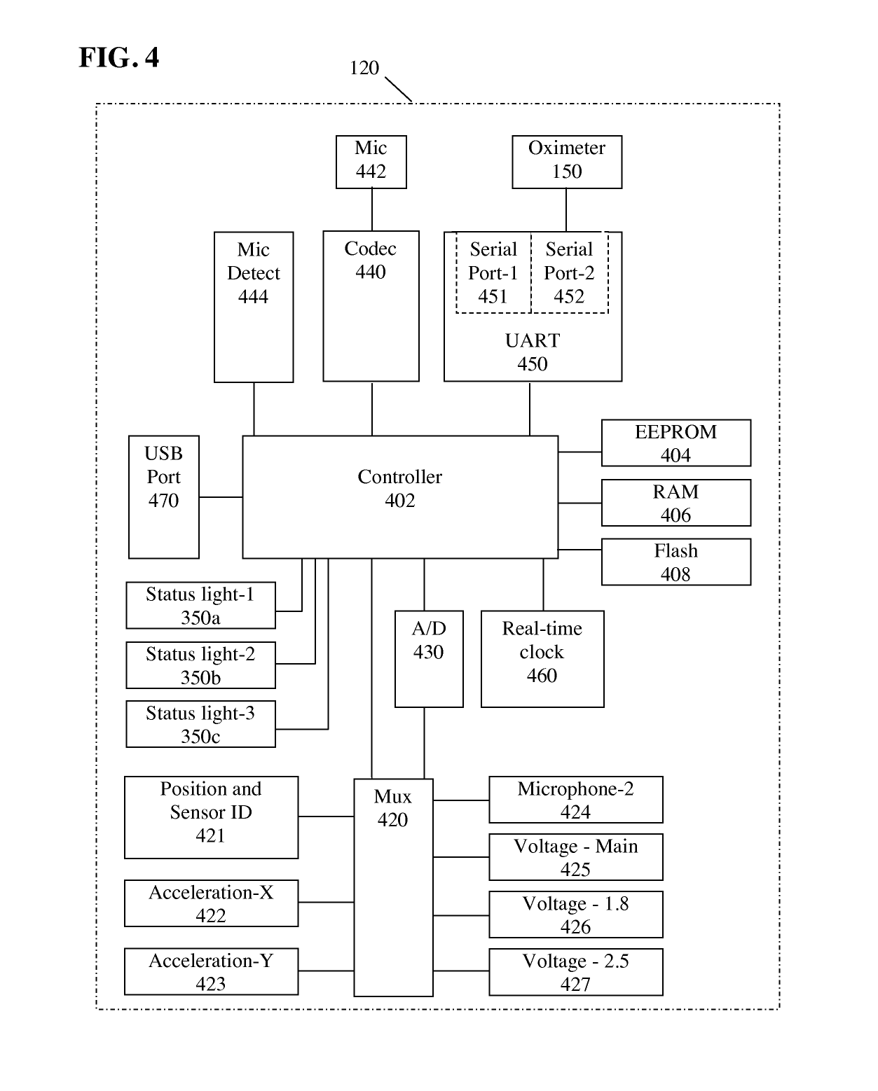

Wrist unit 120 is preferably of a size that is unobtrusive to patient 100. We have found that a wrist unit 120 of size 4.4 inches by 2.6 inches by 0.85 inches (the long axis of wrist unit 120 being parallel to the long axis of the forearm associated with wrist 135) is often sufficiently unobstrusive. These dimensions do not include bracket 310 shown in FIG. 3.

In an alternative embodiment, another cable 140 connects to wrist unit 120 at a junction point 145 and also connects to an oximeter 150. Cable 140 contains one or more electrically conductive means, thereby providing a means for electronic communication between oximeter 150 and wrist unit 120. Oximeter 150 may be of a standard type and may be commercially available. One available brand is the Xpod, offered by Nonin Inc. Oximeter 150 may be attached to the patient in various locations. In the illustrated embodiment, oximeter 150 is removably coupled to a finger of patient 100, preferably to a finger associated with the same arm with which wrist 135 is associated. In many cases cable 140 may be configured to be sufficiently short so that stabilization of the cable's position at attachment point(s) is unnecessary.

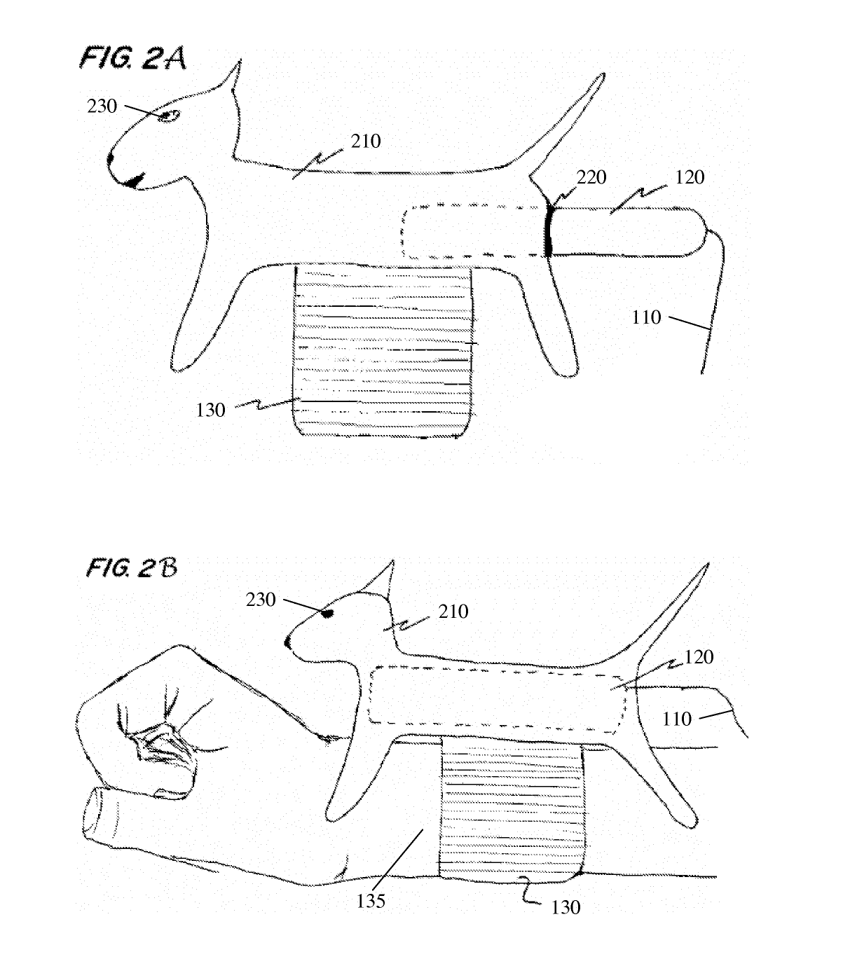

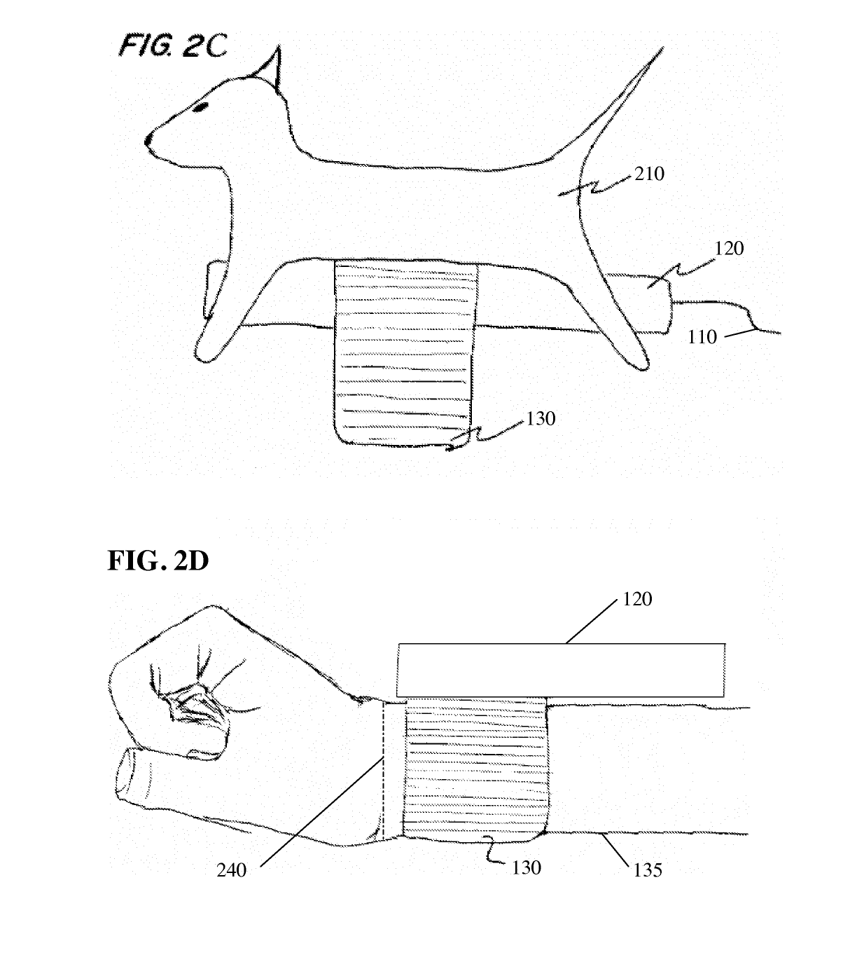

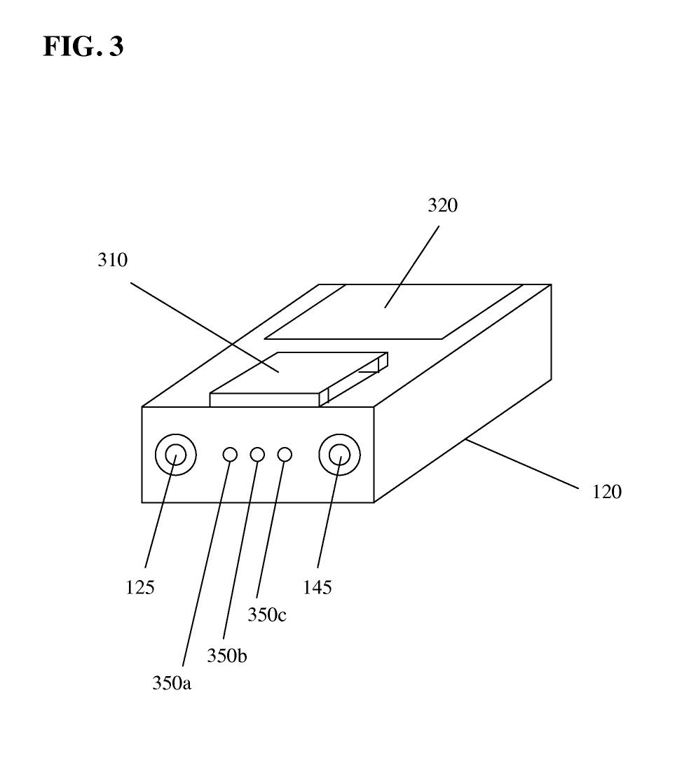

FIGS. 2A, 2B, and 2C show alternate embodiments in which wrist unit 120 is coupled to a “wristy” 210. “Wristy” is the name we give to a soft animal, soft figurine, or other toy that is adapted to receive or otherwise couple to wrist unit 120. Wristy 210 may also be adapted to physically couple to wrist 135 of patient 100 using strap 130. We have discovered that a soft toy in some cases facilitates use of the invention when patient 100 is a child.

Turning attention to FIG. 2A, a wristy figurine 210 has an opening 220 allowing insertion and removal of wrist unit 120 into and out of an internal cavity (not shown) of figurine 210. In one embodiment, opening 220 is zippered or otherwise recloseable, e.g. with hook and loop fasteners, a flap, a button, a snap, and the like. Wrist unit 210 may, therefore, be completely hidden from view of patient 100, although this may not be the case in all embodiments.

In one embodiment, figurine 210 is coupled to strap 130, e.g. by stitching or by hook and loop fasteners. FIG. 2A shows wrist unit 120 partially inserted into figurine 210, and cable 110 attached to wrist unit 120.

FIG. 2B shows wrist unit 120 fully inserted in an internal cavity (not shown) of figurine 210. Cable 110 enters figurine 210 through an opening (not shown in FIG. 2B) that allows entry of wrist unit 120 into figurine 210. In one embodiment cable 110 may have the appearance of a tail of figurine 210. Another embodiment may use a pouch instead of an internal cavity.

Figurine 210 is coupled to strap 130 which, in turn, is coupled to wrist 135. In one embodiment, the physical couplings between wrist unit 120, figurine 210, strap 130, and wrist 135 are sufficiently rigid that motion of wrist 135 is substantially imparted to wrist unit 120, thereby allowing an acceleration sensing means in wrist unit 120 to substantially track the acceleration of wrist 135.

In one embodiment, one or more eyes 230 of figurine 210 display status information, e.g. a blinking light appearing to emanate from the left eye of figurine 210 could indicate normal operation, a blinking light appearing to emanate from both eyes of figurine 210 could indicate a low power state. In another embodiment wrist unit 120 (or figurine 210 itself) may emit sounds such that they appear to come from figurine 210. Such sounds may be appropriate to the appearance of the figurine, e.g. a cat figurine may appear to emit meow-ing sounds. In an alternate embodiment such sounds may include spoken words, e.g. instructions, or a greeting, possibly including the name of patient 100. In one embodiment sounds and lights associated with the figurine may be triggered by an environmental change, e.g. a change in ambient lighting level.

FIG. 2C shows an alternate embodiment of a wristy. Wrist unit 120 is positioned externally to figurine 210 at the ventral surface of figurine 210. Wrist unit 120 and figurine 210 may be coupled to each other or to strap 130 by glue, hook and loop fasteners, stitching, and the like.

In one embodiment, a figurine may be physically associated with tracheal sensor 105. We have discovered that a comfortable site to position strap 130 is at a location on wrist 135 where a wristwatch band is customarily worn. FIG. 2D shows one embodiment in which strap 130 is coupled to wrist 135 just proximal to the extreme distal extent of wrist 135.

Preferably, positioning of wrist unit 120 allows substantially free extension of an associated wrist joint 240 (i.e. the joints at the distal ends of the radius and ulna). We have discovered that positioning wrist unit 120 on the dorsal surface of wrist 135 such that it extends distal to wrist joint 240 results in interference with wrist extension and undesirably adds to the obtrusiveness of wrist unit 120. In one embodiment, strap 130 is positioned near the distal extreme of wrist unit 120 and the maximum distal extent of wrist unit 120 is just proximal enough to wrist joint 240 to allow free extension of the joint.

In one embodiment, wrist unit 120 is of sufficiently small size that it may be worn similarly to a wristwatch, wherein strap 130 is similar to a watchband.

FIG. 3 shows one embodiment of wrist unit 120. For convenience of illustration, wrist unit 120 is positioned upside-down in FIG. 3 with respect to depiction of wrist unit 120 in FIG. 2, such that the top surface in FIG. 3 is the portion that is adjacent to the skin of patient 100 in use. The long axis of wrist unit 120 parallels the long axis of the associated wrist of the patient 100. Wrist unit 120 includes a bracket 310 for coupling to a removable wrist strap 130. Wrist unit 120 includes an internal compartment for batteries (not shown) and a door 320 to the battery compartment. Door 320 may be completely removable or may remain attached when opened, e.g. by one or more hinges.

The junction point 125 between cable 110 and wrist unit 120 in FIG. 1 is shown in more detail in FIG. 3 where cable 110, terminating in a suitable plug such as a bayonet audio plug, can be plugged. In an alternative embodiment, cable 110 is hardwired into wrist unit 120, and port 125 is a conduit.

The junction point 145 between cable 140 and wrist unit 120 in FIG. 1 is shown in more detail in FIG. 3 where cable 140, terminating in a suitable plug, can be plugged. In an alternative embodiment, cable 140 is hardwired into wrist unit 120, and junction point 145 is a conduit. In an alternate embodiment, as above, junction point 145 does not exist and oximeter 150 is not used.

To communicate various status conditions, wrist unit 120 may include one or more status lights 350. FIG. 3 shows three status lights 350a, 350b, and 350c. In one embodiment, different patterns of illumination of status lights 350 convey different status conditions. Such patterns may vary in time as well as in the specific status light(s) that are illuminated. Status lights 350 may be labeled with alphanumeric or iconic legends, e.g. a battery icon, to facilitate attachment of a particular meaning to a particular status light 350. Status lights 350 may be dim when viewed in daylight conditions, so that they are not undesirably bright in a dark environment where patient 100 or a bed-partner of patient 100 may be trying to sleep and thereby potentially interfere with sleep. In an alternative embodiment, wrist unit 120 may include a means to reversibly cover, dim, or otherwise obscure status lights 350. In another embodiment, status lights 350 may function for only a portion of the time associated with sleep, e.g. the first 10 minutes. In another alternative environment, status lights 350 may function only under lit ambient conditions; this approach may require a sensor coupled to status lights 350 that provides signals indicative of ambient light level. In one embodiment, light emanating from status lights 350 is transmitted to one or more eyes 230 of figurine 210.

In one embodiment, wrist unit 120 provides numerous functions, including but not restricted to those provided by a controller, one or more batteries, a memory, and an acceleration sensor.

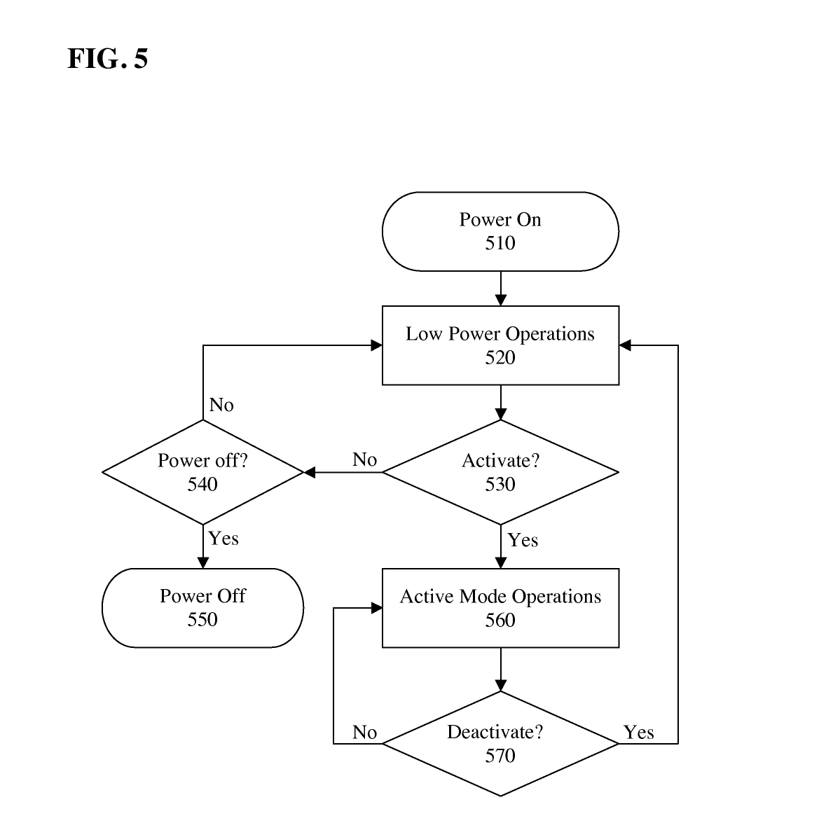

FIG. 4 shows a block diagram representation of certain functions provided by wrist unit 120 in one embodiment. A controller 402 provides input and output functions. Controller 402 may operate on the basis of software codes stored in an electrically erasable programmable read-only memory (EEPROM) 404. Controller 402 may use a random access memory (RAM) 406 for a variety of purposes, for example data-buffering, and may write various types of data to a flash memory 408. Other embodiments are possible, e.g. replacing (or supplementing) flash memory 408 with another type of solid state memory, a hard-disk drive having a small form factor, or a memory mounted on a printed circuit board.

Controller 402 may receive input from a plurality of sources. A microphone 442 coupled to tracheal sensor 105 (not shown in FIG. 4) may provide signals indicative of tracheal sounds of patient 100 to a codec 440. Codec 440 may provide information derived from these signals to controller 402, and may convert analog signals provided by microphone 442 into digital form. Preferably microphone 442 provides at least signals indicative of quiet breathing of patient 100, i.e. tracheal breath sounds. In one embodiment microphone 442 is substantially contained within tracheal sensor 105.

Controller 402 may receive input from a source 444 indicating, e.g., whether microphone 442 is informationally coupled to codec 440, i.e. that codec 440 can receive information from microphone 442. In one embodiment, informational coupling is accomplished as an electrical coupling, e.g. via cable 110 or a wireless link Microphone detection input from source 444 may be valuable if, for example, controller 402 executes different software codes depending on whether or not microphone 442 is informationally coupled to codec 440.

In another embodiment input from source 444 may be indicative of cable 110 being plugged into wrist unit 120. In this embodiment, an informational coupling between microphone 442 and codec 440 may then be inferred with a degree of reliability.

An oximeter 150 coupled to patient 100 may provide signals indicative of oxygen saturation and/or pulse rate to a serial port 452. In one embodiment, serial port 452 and a second serial port 451 may be managed by a universal asynchronous receiver-transmitter (UART) 450. UART 450 may provide information derived from signals provided by oximeter 150 to controller 402.

A real-time clock 460 may provide time-of-day information to controller 402. An analog-to-digital (A/D) converter 440 may provide information to controller 402. In one embodiment, this information may come from one or more of several possible sources 421 through 427. A multiplexer (mux) 420 may select from among these signal sources and provide signals from the selected source(s) to A/D converter 430. A/D converter 430 may convert signals from analog form to digital form and provide signals in digital form to controller 402.

Controller 402 may signal mux 420 which source to select at various times. In some embodiments, mux 420 may select from among the following signals:
a signal 421 derived from a position sensor, indicative of the orientation, with respect to gravity or another acceleration vector, of a portion of the body of patient 100;
a signal 422 derived from an acceleration sensor, indicative of the acceleration of a portion of the body of patient 100 along a particular axis;
a signal 423 derived from an acceleration sensor, indicative of the acceleration of the same portion of the body of patient 100 as in signal 422, preferably along an axis substantially orthogonal to the axis associated with signal 422;
a signal 424 derived from a second microphone, indicative of ambient noise in the environment of wrist unit 120;
a signal 425 indicative of the voltage in a first circuit of wrist unit 120, the circuit having a voltage dependent on the voltage supplied by a power source, e.g. batteries;
a signal 426 indicative of the voltage in a second circuit of wrist unit 120, the circuit having a nominal voltage of 1.8 volts; or
a signal 427 indicative of the voltage in a third circuit of wrist unit 120, the circuit having a nominal voltage of 2.5 volts.

In one embodiment signal 421 may reflect not only orientation of a portion of the body of patient 100, but also an identification code associated with tracheal sensor 105. In this embodiment an analytical means (not shown) may separate orientation information from sensor identification information encoded in data deriving from signal 421. Sensor identification information may, for example, be associated with a particular tracheal sensor 105 or may be indicative of a class of sensors to which a particular tracheal sensor 105 belongs (e.g. adult-sized sensor, pediatric-sized sensor). Sensor identification information may be useful if, for example, an analytical means applies different analyses depending on the sensor or class of sensor used. Sensor identification information may also be useful for tracking and other purposes. In an alternate embodiment, sensor identification information is obtained by controller 402 by a separate signal from orientation information.

In one embodiment, orientation information is obtained from an apparatus or method as described in co-pending U.S. patent application Ser. No. 10/721,115. In an exemplary embodiment, tracheal sensor 105 may include a position sensor, microphone 442, and sensor identification means.

One or more acceleration sensors may, in one embodiment, be comprised of one or more electro-mechanical elements within wrist unit 120. The acceleration sensor(s) may be of a standard type and may be commercially available. One available brand is the ADXL202E model from Analog Devices, Inc. This particular brand consists of one solderable component that provides two signals, indicative of acceleration along substantially orthogonal axes. When wrist unit 120 is substantially rigidly coupled to wrist 135, acceleration sensor(s) within wrist unit 120 may be interpreted as measuring acceleration of wrist 135. (A technique known as actigraphy often measures acceleration of a patient's wrist.) In one embodiment, acceleration sensors are positioned to yield signals indicative of wrist 135 movement along a side-to-side axis and an up-down axis.

In one embodiment a second microphone (not shown) is positioned within wrist unit 120.

One or more additional sensors, providing signals indicative of various physiological and/or environmental parameters, may be added in various embodiments. In some embodiments such sensors may informationally couple to mux 420. For example, in various embodiments:
a sensor positioned on a finger of patient 100 may provide a signal indicative of arterial tone or arterial cross-sectional area (e.g. a peripheral artery such as in a finger);
a sensor positioned about the trunk of patient 100 may provide a signal indicative of thoracic or abdominal respiratory effort (a respiratory effort sensor may, for example, be based on a circumferential band or on plethysmography);
a sensor positioned on a lower extremity may provide a signal indicative of movement of that lower extremity, such that the presence or absence of restless legs syndrome may be assessed;
a sensor may be added to provide a signal indicative of jaw-muscle activity, such that the presence or absence of bruxism may be assessed (a jaw-muscle sensor may be, for example, an electromyographic sensor or a microphone).

In one embodiment, electrically sensitive sensors may be incorporated into portions of tracheal sensor 105 and strap 130 (or possibly wrist unit 120) in contact with skin of patient 100, such that a signal indicative of cardiac electrical activity is provided. In such a case it may be preferable to couple wrist unit 120 to the wrist of patient 100 ipsilateral to the patient's heart (i.e. normally the left wrist), so as to better capture cardiac electrical activity. In an embodiment where, instead of wrist unit 120 coupling to wrist 135 via strap 130, wrist unit 120 is adhesively coupled to an upper extremity of patient 100, an electrically sensitive sensor may be associated with the coupling such that a signal indicative of cardiac electrical activity is provided. A wrist location is preferable for collecting cardiac-related electrical signals because of its generally weaker myo-electrical signal as compared to a hand location.

In one embodiment serial ports 451 and 452 allow controller 402 to communicate with devices having a serial interface. In this embodiment controller 402 may be programmed by uploading software codes to controller 402 via serial port 451. Controller 402 may use serial port 451 and 452 for output and input.

In one embodiment controller 402 may communicate with a device, for example, a computer, personal digital assistant (PDA), or other data processing device (not shown) via USB port 470.

In one embodiment controller 402 may output information using status lights 350a through 350c. Controller 402 may illuminate and de-illuminate signal lights 350a through 350c individually.

As above, tracheal sensor 105, wrist unit 120, and, if present, oximeter 150 collectively comprise system 10 in FIG. 1.

In some embodiments one or more power sources (not shown) may supply energy to components of system 10. In one embodiment, controller 402 may cut-off and restore power to one or more components at various times, possibly conserving power as a result. In various embodiments, different power sources may be used, for example, two size-AA batteries may supply electrical power to components of system 10 for operation. In another embodiment an additional battery may be dedicated to supplying power to real-time clock 460, so the operation of the clock is not interrupted if the two AA batteries are removed.

In one embodiment, cable 110 may contain three conductive means (not shown). The conductive means may transmit energy from wrist unit 120 to tracheal sensor 105 and return information from tracheal sensor 105 to wrist unit 120. In some embodiments the conductive means may informationally couple with mux 420, codec 440, and controller 402, and electrically couple with a power source (not shown) in wrist unit 120.

FIG. 6 shows an embodiment in which the functions of wrist unit 120 are divided between a PDA-adapter 600 and a PDA 601. PDA 601 may be commercially available. Two available brands are the Tungsten™ T5 and the Treo™ 650 Smartphone, respectively, offered by PalmOne Inc.

In one embodiment, PDA-adapter 600 docks to a utility port of PDA 601 and through this port may obtain power and exchange information with PDA 601. Components within PDA-adapter 600 may be reduced in comparison with wrist unit 120 because of the functions assumed by components of PDA 601. For example, flash memory 408 may be unnecessary in PDA-adapter 600 because PDA 601 may provide memory of sufficient capacity. As an additional example, a second serial port may be unnecessary in PDA-adapter 600 if controller 402 can be programmed by downloading software codes from PDA 601. As still an additional example, USB port 470 may be included in PDA. In some embodiments it may be possible to omit codec 440 and microphone detection signal 444 in favor of a simpler connection between microphone 442 and mux 420.

In one embodiment inputs from tracheal sensor 105 pass through cable 110 and into PDA-adapter 600. Oximeter 150 may also provide input to PDA-adapter 600 through serial port 452 and UART 450. In an embodiment wherein PDA-adapter 600 is substantially rigidly coupled to wrist 135, acceleration sensors 422 and 423 may provide signals indicative of acceleration of wrist 135. An embodiment may include a smaller number of status lights 350 in PDA-adapter 600 as compared to wrist unit 120, as in FIG. 6 that shows only one status light 350a. Controller 402 may operate similarly with respect to EEPROM 404, RAM 406, mux 420, and A/D converter 430 as in wrist unit 120.

In one embodiment both PDA-adapter 600 and PDA 601 are coupled to wrist 135 such that tethering does not appreciably occur.

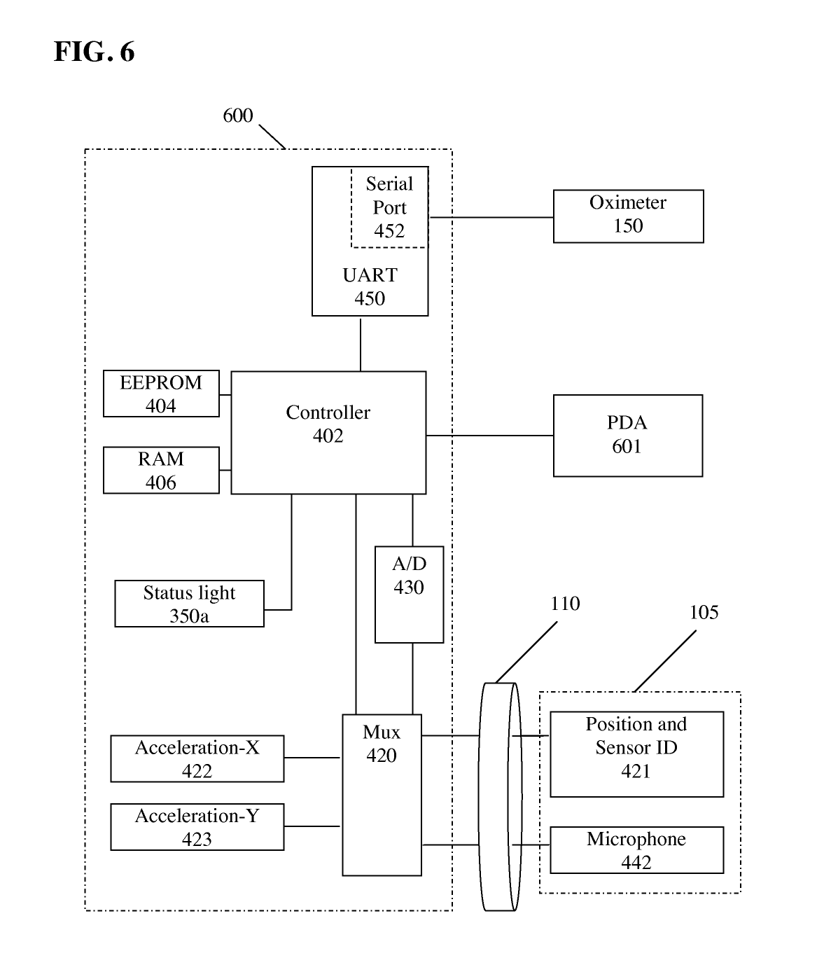

In one embodiment system 10 may operate in a plurality of power modes, e.g., a low-power mode and an active mode, and transition between them. FIG. 5 shows a flow diagram of possible power modes and transitions according to one such embodiment.

When power is initially applied at step 510, system 10 may perform certain start-up functions (not shown), enter a low-power mode, and perform functions at step 520 associated with that mode, for example, illumination of status light 350a in a particular pattern. Start-up functions may include, for example, internal diagnostics.

At various times in low-power mode, system 10 may determine at step 530 whether a transition to active mode is indicated. A transition to active mode may be indicated if, for example, a specified time-of-day has been reached in comparison with information from real-time clock 460, or if signal source 444 has assumed a particular value indicating that microphone 442 is coupled to codec 440. Other indications are possible.

If a transition to the active mode is not indicated, system 10 may determine at step 540 whether to perform a controlled power-off, step 550, of all its components. Power-off step 550 may be indicated if, for example, a voltage level within wrist unit 120 has reached a critically low level. If power-off is not indicated, then system 10 may return to the low-power operation of step 520.

If transition to the active mode is indicated, active mode operations may begin as defined in step 560, e.g. acquiring data, checking available memory capacity, writing data to RAM 406 and/or flash 408 memory, illuminating status light 350b in a particular pattern, and the like. Data may be acquired from one or more sources, e.g. A/D converter 430, codec 440, UART 450, and real-time clock 460. In one embodiment, data are buffered in RAM memory 406 before being written to flash memory 408.

At various times in active mode, system 10 may determine at step 570 whether a transition to low-power mode is indicated. A transition to low-power mode may be indicated if, for example, microphone 444 is no longer coupled to codec 440, flash memory 408 is full, or a power level is low.

If a transition to the low-power mode is not indicated, system 10 continues active mode operations. If a transition to low-power mode is indicated, system 10 begins the low-power operations of step 520.

Other modes and mode inter-relationships are possible. In general, system 10 will have, at a minimum, an active mode, during which data are acquired and stored, and a powered-off mode. A mode to transfer data out of system 10 may also be desirable.

In one method, patient 100 and/or a caretaker of patient 100 applies system 10 to patient 100 shortly before patient 100 goes to bed to sleep. This may include attachment of tracheal sensor 105 to a peri-tracheal location of patient 100, coupling of wrist unit 120 to wrist 135, and possibly application of oximeter 150 to patient 100. Optionally, patient 100 or caretaker fastens cable 110 at one or more fixation points 115, preferably within a few minutes of the time system 10 is applied.

In one embodiment, patient 100 or a caretaker applies power to system 10 shortly before patient 100 goes to bed to sleep (step 510). In one embodiment, this may be done by patient 100 or a caretaker engaging an on/off switch on wrist unit 120 from the “off” condition to the “on” condition. In another embodiment, patient 100 or caretaker may pull an insulating tab from wrist unit 120, e.g. in the vicinity of battery compartment door 320, thereby enabling power to flow from a power source (not shown in FIG. 1) to one or more components of system 10. These embodiments assume that batteries have previously been inserted into system 10. In an alternate embodiment power is applied by inserting one or more batteries into wrist unit 120, without utilization of a discrete on/off switch.

In another embodiment, patient 100 or a caretaker plugs cable 110 into a plug at junction point 125 shortly before patient 100 goes to bed to sleep, resulting in a transition from low-power operations 520 to active operations 560.

In some embodiments, status lights 350a through 350c may communicate information to patient 100 or a caretaker that system 10 is functioning correctly in active mode.

After a time, during which patient 100 has normally slept or attempted to sleep, patient 100 or a caretaker may remove all elements of system 10 from patient 100. In one embodiment, patient 100 or a caretaker then remove batteries from wrist unit 120. In another embodiment, an on/off switch is toggled from “on” to “off.” In still another embodiment cable 110 is removed from the plug at junction point 125 and system 10 enters a low-power mode. In yet another embodiment, system 10 continues in an active mode until flash memory 408 is full or power reaches a certain threshold.

In general, after this period of sleep or attempted sleep, flash memory 408 contains physiological data acquired from patient 100 during the time system 10 was in an active mode.

In one embodiment, patient 100 or a caretaker return system 10 to a health care provider for analysis of the collected physiological data. In another embodiment, patient 100 or a caretaker retain system 10 for analysis of collected physiological data.

In some embodiments system 10 may be docked via USB port 470 and a USB cable to an external computer or other processing device (not shown) having a USB port. In one embodiment, flash memory 408 appears in the external computer's interface as a volume (or, “drive”), and collected data appear as a file in the volume. This allows transfer of data to and from flash memory 408 and the external computer via controller 402. In one embodiment, a wireless link comprises at least a portion of a coupling between controller 402 and an external device.

Thus, it is seen that system 10 is capable of collecting, recording, and transferring information related to one or more physiological parameters associated with a sleep period of patient 100. In some embodiments, analysis of the collected physiological data may occur on a computer. The analysis may use a plurality of physiological data types, e.g. movement, position, oxygenation, pulse rate, and sound, to assess breathing of patient 100. In one embodiment, the analysis may provide a plurality of assessments related to the breathing of patient 100. An assessment may be derived from consideration of information from one type of sensor signal, or derived from considerations of more than one type of sensor signal.

For example, consideration of information obtained from microphone 442 in one embodiment may provide assessments of respiratory rate, apnea events, hypopnea events, type of apnea/hypopnea events (e.g. obstructive vs. central), snoring, duration of respiratory phases, micro-apnea density (relative and absolute), respiratory regularity, respiratory self-similarity, state of consciousness, and signal quality. In one embodiment, state of consciousness may be identified as wakefulness, sleep, rapid-eye-movement (REM) sleep, or non-REM sleep. Because cardiovascular sounds are sometimes present in tracheal sound, consideration of information obtained from microphone 442 may provide an assessment of cardiovascular state such as heart rate and timing between first heart sound (S₁) and second heart sound (S₂). Other sonic phenomena may be assessed by consideration of information obtained from microphone 422, including, but not limited to talking, sighing, coughing, swallowing, sneezing, wheezing, stridor, yawning, grunting, grinding (of teeth), hiccoughing, belching, and position-changing (which generally includes significant non-respiratory sounds).

In some embodiments, information obtained from microphone 442 may be combined with information obtained from one or more other sensors to provide an improved assessment. For example:
apnea and/or hypopnea events may be assessed by considering information derived from microphone 442 and oxygen saturation information from oximeter 150;
apnea and/or hypopnea events may be assessed by considering information derived from microphone 442 an arterial tone or cross-section sensor (not shown);
sleep fragmentation (and tranquility) may be assessed by combining information about apnea and hypopnea events with information about arm movement derived from accelerometer signals 422 and 423;
information derived from electrocardiographic sensors (not shown), an arterial tone or cross-section sensor (not shown), and/or oximeter 150 may facilitate identification of heart sounds monitored by microphone 442;
if information derived from microphone 442 indicates that tracheal sensor 105 was improperly positioned over a certain time period, position signal 421 may be reported as “unusable” for that time period;
information from electrocardiographic sensors (not shown) about cardiac rhythm may be combined with timing of apneas, hypopneas, or other types of events, to determine the degree by which cardiac rhythm influences the occurrence or duration of these events; and
information derived from position signal 421 may be used to predict response of patient 100 to positional therapy for snoring, sleep apnea, or other condition. For example, if a patient snored X % of the 8 hours during which physiological information was obtained and snored Y % of the 3 hours he was on his back, then subtraction and normalization of these results could provide an estimate of the percentage of time he would snore if no back-sleeping occurred.

In some embodiments, information obtained from a plurality of sensors other than microphone 442 may be combined to provide improved assessment. For example, combining information derived from acceleration signals 422 and 423 may provide an overall assessment of wrist 135 movement. As a further example, assessment of body position may be improved by combining information derived from position signal 421 and acceleration signals 422 and 423.

In some embodiments information about relative timing of cardiovascular events and, possibly, an assessment of certain aspects of cardiopulmonary and/or cardiovascular function, may be provided by combining information derived from electrocardiographic sensors (not shown), heart sounds monitored by microphone 442, an arterial tone or cross-section sensor (positioned, for example, on an extremity), and/or oximeter 150.

Thus, it is seen that system 10 and associated methods may provide information amenable for use in a plurality of assessments of patient 100. Such assessments may relate to the breathing of patient 100, possibly during sleep, but the invention is not limited to breathing assessments only. Diagnostic and/or management decisions in the care of patient 100 may be based, in whole or in part, on the results of such assessments. Systems and methods may suitably report or visualize physiological information collected by system 10, or assessments based thereon, so as to facilitate such decisions.

In one embodiment, information derived from data collected by system 10 may be stratified by body position (e.g. facing up, down, left, right) and/or sleep stage (e.g. wakefulness, REM sleep, and sleep stages 1, 2, 3, 4), and so visualized or reported (as, for example, a position-specific snoring index or apnea-hypopnea index).

It should be noted that the above sequence of steps is merely illustrative. The steps can be performed using computer software or hardware or a combination of hardware and software. Any of the above steps can also be separated or be combined, depending upon the embodiment. In some cases, the steps can also be changed in order without limiting the scope of the invention claimed herein. One of ordinary skill in the art would recognize many other variations, modifications, and alternatives. It is also understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A system for recording one or more parameters of a patient, comprising:
a microphone coupled to a peri-tracheal portion of the patient's body and configured to provide a signal indicative of at least quiet breathing of the patient; and
a memory configured to store information derived from the signal indicative of at least quiet breathing of the patient; and
a controller configured to write the information into the memory; and
a power source configured to supply electrical energy to at least one of the microphone, the memory, or the controller; and
a housing containing one or more of the controller or the memory or the power source, coupled to a non-peri-tracheal portion of the patient's body such that the housing moves substantially in concert with said non-peri-tracheal portion of the body.
